(19) United States
(12) Patent Application Publication
(10) Pub. No.: US 2022/0006981 A1
(43) Pub. Date: Jan. 6, 2022

(54) METHOD OF AUTOMATIC IMAGE FREEZING OF DIGESTIVE ENDOSCOPY

(71) Applicant: Wuhan Endoangel Medical Technology Co., Ltd., Wuhan (CN)

(72) Inventors: Chao LI, Wuhan (CN); Jinlong LIU, Wuhan (CN); Qiwei LIU, Wuhan (CN); Shan HU, Wuhan (CN)

(21) Appl. No.: 17/138,868

(22) Filed: Dec. 30, 2020

(30) Foreign Application Priority Data
Jul. 1, 2020 (CN) 202010620041.6

(51) Int. Cl. H04N 7/18; G06K 9/62; H04N 5/262; G06T 3/40; G06K 9/00; A61B 1/06; A61B 1/273

(52) U.S. Cl. CPC H04N 7/188; G06K 9/6215; H04N 5/2628; A61B 1/2736; G06K 9/00744; A61B 1/0638; G06T 3/4007

(57) ABSTRACT

A method of automatic image freezing of digestive endoscopy based on a perceptual hash algorithm includes: analyzing a video streaming of digestive endoscopy acquired by digestive endoscopy imaging system into image data; calculating a similarity between an image at t point in time and images of first n frames, to obtain a weighted similarity k of the image; and comparing the weighted similarity k of the image at t point in time with a freezing boundary l, and triggering an instruction of image freezing when the k reaches l to obtain the clear images with the best visual field from the video streaming of digestive endoscopy.

### CROSS-REFERENCE TO RELAYED APPLICATIONS

Pursuant to 35 U.S.C. § 119 and the Paris Convention Treaty, this application claims foreign priority to Chinese Patent Application No. 202010620041.6 filed Jul. 1, 2020, the contents of which, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND

This disclosure relates to the field of medical image processing technology, and more particularly to a method of automatic image freezing of digestive endoscopy based on a perceptual hash algorithm.

Digestive endoscope is a set of equipment for the diagnosis and treatment of digestive diseases by means of images obtained from the digestive tract or by ultrasound and X-ray images of the digestive tract and digestive organs. In the process of digestive endoscopy, the digestive endoscopy imaging system (combined with digestive endoscopy, display system and computer workstation) inputs the digestive endoscopy image into the computer for digital processing, and displays the output image in real time, which can be used for image freezing, acquisition and storage. Among them, image freezing is a common basic function commonly used in the process of digestive endoscopy. Endoscopists freeze real-time images by pressing the “freeze” button of digestive endoscope equipment to obtain static images for careful observation. For endoscopists with low operation level and slow reaction speed, when freezing the image manually, it is possible to obtain images that are fuzzy and miss the best view angle.

### SUMMARY

The disclosure provides a method of automatic image freezing of digestive endoscopy based on a perceptual hash algorithm.

Specifically, the method of automatic image freezing of digestive endoscopy based on a perceptual hash algorithm comprises:
1) analyzing a video streaming of digestive endoscopy acquired by digestive endoscopy imaging system into image data;
2) calculating a similarity between an image at t point in time and images of first n frames, to obtain a weighted similarity k of the image;
3) comparing the weighted similarity k of the image at t point in time with a freezing boundary l, and triggering an instruction of image freezing when the k reaches l to obtain static images from the video streaming of digestive endoscopy.

In a class of this embodiment, in 1), the method further comprises removing fuzzy invalid frame images, cropping clear images, reducing a size of cropped images, retaining the image structure information, and converting the cropped images into gray scale images.

In a class of this embodiment, in 1), bicubic interpolation is adopted to reduce the size of the cropped images.

In a class of this embodiment, in 1), a calculation formula of converting the cropped images into the gray scale images is as follows:

Gray=0.30*R+0.59*G+0.11*B;

where R, G and B respectively represent information values of red light, green light and blue light.

In a class of this embodiment, in 1), the Gray-scale value of adjacent pixels in each line of the gray image are compared. If the gray value of the previous pixel is greater than that of the latter pixel, the dHash value is set to “1”, if not, the dHash value is set to “0”.

In a class of this embodiment, in 2), the similarity between different images is calculated by calculating a Hamming distance between different images.

In a class of this embodiment, in 2), the Hamming distance between different images refers to a number of digits required to change dHash values corresponding to a first image to dHash values corresponding to a second image. For example, the Hamming distance between 0110 and 1111 is 2.

In a class of this embodiment, in 2), a formula for calculating the similarity between a current image and the first n frames is as follows:

Sim=100*(64−d(x,y))/64;

where the d (x, y) is the Hamming distance between different images, d (x, y)=Σx⊕y, x and y are the dHash values corresponding to different images, and ⊕ is exclusive OR.

In a class of this embodiment, in 3), the freezing boundary l is obtained by analyzing the video of manually freezing image by endoscopist during the digestive endoscopy.

In a class of this embodiment, in 3), when k≥l, it is judged that the image is frozen at t point in time, then the image freezing command is triggered at t time point, that is, the image can be frozen without endoscopist's operation of “freezing”; when k<l, it is judged that t point time is not a frozen image, then the command of freezing image at t point time is not triggered.

The following advantages are associated with the method of automatic image freezing of digestive endoscopy of the disclosure. Using this method, when endoscopists need to carefully examine the image of a certain visual field, they only need to stop the movement of the endoscopic body to keep the visual field unchanged. Then the images can be automatically determined as frozen images. There is no need for the endoscopists to manually operate the “freeze” button, thus reducing the workload of the endoscopists. The system automatically executes the freezing instruction, which can avoid the deviation of visual field or loss of effective information of frozen images due to slow reaction or unskilled operation, thus effectively acquiring the clear images with the best visual field.

### BRIEF DESCRIPTION OF THE DRAWINGS

FIG. 1 is a flow chart of a method of automatic image freezing of digestive endoscopy according to one embodiment of the disclosure;

FIG. 2 is a schematic diagram of scaling images by bicubic interpolation according to one embodiment of the disclosure;

FIG. 3 is s schematic diagram of a pixel (x, y) in a target interpolation graph which is the closest mapping point in the original images according to one embodiment of the disclosure; and

FIG. 4 is a gray scale image according to one embodiment of the disclosure.

### DETAILED DESCRIPTION

To further illustrate the disclosure, embodiments detailing a method of automatic image freezing of digestive endoscopy based on a perceptual hash algorithm are described below. It should be noted that the following embodiments are intended to describe and not to limit the disclosure.

Image structure information: refers to the hue change and position arrangement of each pixel in the image.

Gray-scale value: the black tone is used to represent the color of image, gray-scale is the brightness of pixel is divided into 256 grades from 0 to 255. Gray-scale value is the number from 0 to 255, 0 represents black, and 255 represents white.

Gray scale image: the image is composed of every pixel represented by Gray-scale value.

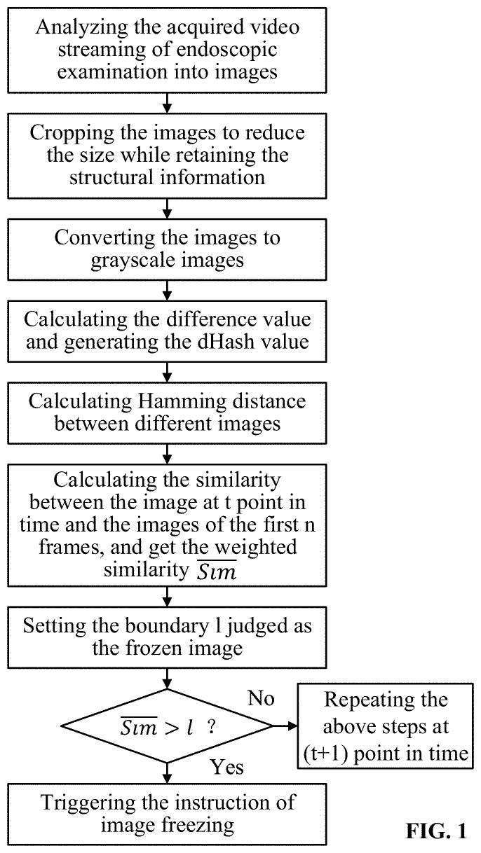

As shown in FIGS. 1-4, the disclosure provides a method of automatic image freezing of digestive endoscopy based on a perceptual hash algorithm, the method comprising:

S1. analyzing a video streaming of digestive endoscopy acquired by digestive endoscopy imaging system into image data;

S2. calculating a similarity between an image at t point in time and images of first n frames, to obtain a weighted similarity k of the image; and

S3. comparing the weighted similarity k of the image at t point in time with a freezing boundary l, and triggering an instruction of image freezing when the k reaches l to obtain the clear images with the best visual field from the video streaming of digestive endoscopy.

### Example 1

S1. Obtaining the video streaming of digestive endoscopy through the digestive endoscopy imaging system, and analyzing the video streaming into images (30 frames per second). Then remove fuzzy invalid frame images and take 10 of them;

S2. Cropping the valid frame images to 360*360 pixels, further reducing the size of cropped images, and only retaining the structural information of images;

An image with 360*360 pixel has more than 100,000 pixels, containing a huge amount of information, and many details need to be processed. Therefore, the image is required to be scaled to a very small size. The purpose is to remove the details of the image, and only retain the basic information such as structure, light and shade, and discard the differences caused by different sizes and proportions.

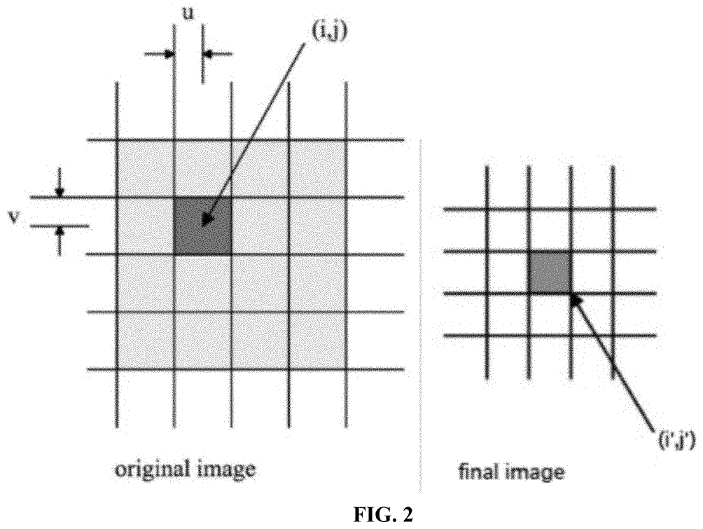

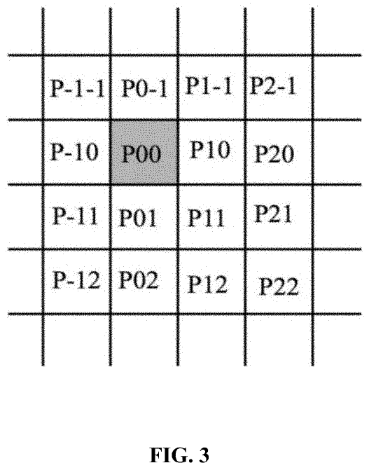

The bicubic interpolation is adopted to scale the image. Although the calculation is large, the quality of the scaled image is high and the image is not easy to be distorted. According to FIG. 2 and the mathematical expression of the bicubic interpolation, it can be seen that the pixel value corresponding to the coordinate point (i, j) in the reduced image after the interpolation is the sum of the weight convolution of the adjacent 16 pixel points at (i, j) in the original image. P00 in FIG. 3 represents a pixel (x, y) in the target interpolation graph which is the closest mapping point in the original images. If the expression of pixel value of each coordinate point of (i, j) in the original image is f (i, j), then the pixel value of corresponding coordinate after interpolation is F (i′, j′), it can be obtained by the following formula:

F(i′,j′)=Σ_{row=-1}²Σ_{col=-1}²f(i+row,j+col)S(row−v)S(col−u)

where v represents the deviation of the number of rows, u represents the deviation of the number of columns; row represents a row, col represents a column; S(x) represents the interpolation expression, comprising common expressions based on trigonometric values, Bell distribution, and B-spline curve, which can be selected according to different needs. The Bell distribution expression is selected in the embodiment of the disclosure.

In order to better calculate the dHash value of the converted images, the embodiment of the disclosure reduces the images to 9*8, a total of 72 pixels.

Converting the images to gray scale images;

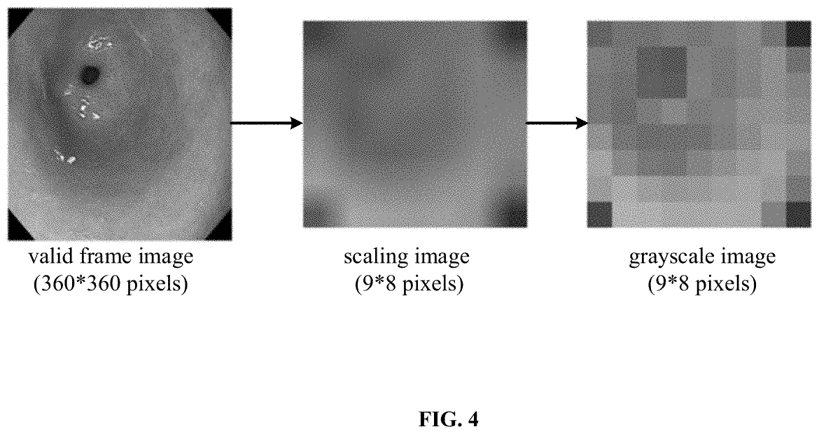

The reduced images are color and consists of RGB values represented as (R, G, B). R, G and B are the information values of red light, green light and blue light respectively. The larger the value is, the brighter the color is, while the smaller the value is, the darker the color is. For example, white represents (255,255,255) and black represents (0,0,0). In general, there is little relationship between image similarity and color. Therefore, the image is processed into gray scale image to reduce the complexity of later calculation, referring to the final obtained gray scale image with 9*8 pixel in FIG. 4. RGB values can be converted to Gray-scale value (represented only by an integer between 0 and 255).

The weighted average method is adopted: due to the different sensitivity of human eyes to red, green and blue, different weights are given to each pixel of the images to calculate the grays values. The formula is as follows:

Gray=0.30*R+0.59*G+0.11*B

Comparing the gray difference of pixels of gray scale images, calculate the difference values, and generate the dHash values of images.

The gray scale images have 9 pixels per row for a total of 8 rows. Comparing the difference between two adjacent pixels in each row, and each row generates eight difference values. If the gray value of the previous pixel is greater than that of the latter pixel, the difference value is set to “1”, if not, the difference value is set to “0”. Then the calculated difference values of the pixels are compared from top to bottom and from left to right, and splice them into 64-bit binary string in order, which is the dHash values of the images.

### Example 2

The example is basically the same as that in Example 1 except the following descriptions.

In S2, the similarity between different images is calculated by calculating the Hamming distance between different images. The Hamming distance between different images represents the number of digits required to change dHash values corresponding to image A to dHash values corresponding to image B. The formula to calculate the similarity between the current image and the first n frames is:

Sim=100*(64−d(x,y))/64;

where the d (x, y) is the Hamming distance between different images, d (x, y)=Σx⊕y, x and y are the dHash values corresponding to different images, and ⊕ is exclusive OR.

Calculating the Hamming Distance Between Different Images;

Hamming distance represents the number of different characters in the corresponding position of two equal length strings, which in dHash is to take the binary dHash value of two images to exclusive OR and calculate the digit of “1” of the exclusive OR result, that is, the digit with different binary dHash values. The Hamming distance between the strings x and y is defined as d (x, y):

d(x,y)=Σx⊕y

⊕ is exclusive OR; x and y are the dHash values corresponding to different images.

S6. Comparing the dHash values of the image at t point in time and the images of the first 9 frames to obtain the overlap rate of the current image and the images of the first 9 frames respectively, namely, the similarity. The calculation formula of similarity Sim of two images is Sim=100*(64−d (x, y))/64. And the weighted similarity of image at t point in time is obtained,

$$\overline{Sim} = {\sum }_{i = 1}^{9} \frac{i}{45} * {Sim}_{i} ,$$

Simᵢ represents the similarity between the image t point in time and the images of the first i frames (i value range is 1-9).

### Example 3

The example is basically the same as that in Example 2 except the following descriptions.

The freezing boundary l of the weighted similarity is set by analyzing the video of manually freezing image by endoscopist during digestive endoscopy.

The weighted similarity S̅i̅m̅ of the image at t point in time was compared with the cutoff l. When S̅i̅m̅≤l, the image is judged as frozen image at t point in time and triggering an instruction of image freezing at t point in time; When S̅i̅m̅>l, the image can't be judged as frozen image at t point in time and can't trigger the instruction of image freezing at t point in time, and repeat the above steps at the next (t+1) point in time.

This technical scheme is used to replace the operation of manually freezing image, which can not only effectively obtain the clear image of the best visual field, but also reduce the workload of endoscopist. The core is how to trigger the instruction of image freezing. Based on the habit of human operation, when endoscopist want to capture static images for freezing operation, they will try their best to keep the endoscopic body and the examination area to remain relatively static. The similarity of sequent frames in the output videos is very high. The perceptual hash algorithm (hereinafter referred to as PHA) is a kind of hash algorithm, which is mainly used to search similar images. PHA is a general name of a class of hash algorithm, whose function is to generate the “fingerprint” string of each image and compare the fingerprint information of different images to judge the similarity of images. The closer the results are, the more similar the images are. PHA comprises average hash (aHash), perceptual hash (pHash) and different hash (dHash).

From the above analysis, PHA is used to analyze and calculate the similarity of the adjacent frames in the per unit time of the digestive endoscopic images. The higher the similarity is, the more likely the image will be frozen. When the similarity reaches the preset boundary, it can be considered as freezing operation, which can automatically issue the freezing instruction, and complete the subsequent process.

The disclosure enables endoscopists only need to stop the movement of endoscopic body to keep the visual field unchanged when they need to carefully examine the image of a certain visual field. Then the images can be automatically determined as frozen images. There is no need for the endoscopists to manually operate the “freeze” button, so as to reduce the workload of the endoscopists. The system automatically executes the freezing instruction, which can avoid the deviation of visual field or loss of effective information of frozen images due to slow reaction or unskilled operation, so as to effectively obtain the clear images with the best visual field.

It will be obvious to those skilled in the art that changes and modifications may be made, and therefore, the aim in the appended claims is to cover all such changes and modifications.

What is claimed is:

1. A method, comprising:
1) analyzing a video streaming of digestive endoscopy acquired by a digestive endoscopy imaging system into image data;
2) calculating a similarity between an image at t point in time and images of first n frames, to obtain a weighted similarity k of the image; and
3) comparing the weighted similarity k of the image at t point in time with a freezing boundary l, and triggering an instruction of image freezing when the k reaches l to obtain clear images with a best visual field from the video streaming of digestive endoscopy.

2. The method of claim 1, wherein in 1), the method further comprises removing fuzzy invalid frame images, cropping the clear images, reducing a size of cropped images, retaining image structure information, and converting the cropped images into gray scale images.

3. The method of claim 2, wherein in 1), bicubic interpolation is adopted to reduce the size of the cropped images.

4. The method of claim 2, wherein in 1), a calculation formula of converting the cropped images into the gray scale images is as follows:
Gray=0.30*R+0.59*G+0.11*B;
where R, G and B respectively represent information values of red light, green light and blue light.

5. The method of claim 2, wherein in 1), Gray-scale value of adjacent pixels in each line of a gray image are compared; if a Gray-scale value of a previous pixel is greater than that of a latter pixel, a dHash value is set to “1”, if not, the dHash value is set to “0”.

6. The method of claim 1, wherein in 2), the similarity between different images is calculated by calculating a Hamming distance between different images.

7. The method of claim 6, wherein in 2), the Hamming distance between different images refers to a number of digits required to change dHash values corresponding to a first image to dHash values corresponding to a second image.

8. The method of claim 7, wherein in 2), a formula for calculating the similarity between a current image and the first n frames is as follows:
Sim=100*(64−d(x,y))/64;
where d (x, y) is the Hamming distance between different images, d (x, y)=Σx⊕y, x and y are the dHash values corresponding to different images, and ⊕ is exclusive OR.

9. The method of claim 1, wherein in 3), the freezing boundary l is obtained by analyzing a video of manually freezing image by an endoscopist during the digestive endoscopy.
